(19) United States
(12) Patent Application Publication
(10) Pub. No.: US 2010/0312138 A1
(43) Pub. Date: Dec. 9, 2010

(54) Determining and/or Monitoring Physiological Conditions in Mammals

(76) Inventor: Philip George Chelf Regas, Aurora, CO (US)

Correspondence Address: James L. Wilcox, Esq.;Joseph Aceto, Esq., 1767 Route 313, Perkasie, PA, 18944, US

(21) Appl. No.: 12/480,837

(22) Filed: Jun. 9, 2009

(51) Int. Cl. A61B 10/00

(52) U.S. Cl. 600/551

(57) ABSTRACT

Improvements in a diagnostic system for determining and/or monitoring a physiological condition in a female mammal are disclosed. The diagnostic system includes hardware and software, and comprises algorithms for the recognition of predetermined patterns of data values, said algorithms being applied to said current daily data value and said stored daily data values by said microprocessor means, said microprocessor means controlling said display means to display characters representing one of several states of the mammal in response to recognition of a corresponding one of said predetermined patterns, wherein the algorithms are capable of determining, for example and substantially automatically, variables that can be used to define the beginning of the fertile cycle of the mammal, the end of the fertile cycle of the mammal and the most fertile day of the mammal. The improvements of the present invention involve a novel user interface providing display of the physiological condition over time to the user.

### BACKGROUND OF THE INVENTION

1. Technical Field

This invention relates generally to the field of determining and/or monitoring physiological conditions in mammals, and in particular relates to electronically sensing, predicting and confirming that such physiological conditions exist in female mammals. The invention, in a preferred embodiment, also particularly relates to improved means, methods and apparatus for predicting and confirming ovulation in human females providing advanced capabilities over those previously known.

2. Background Art

While the present invention can he applied to determining and/or monitoring various physiological conditions in mammals, such as those conditions disclosed in U.S. Pat. No. 6,364,844 to Regas, et al., issued Apr. 2, 2002, which is incorporated herein by reference, in particular with respect to predicting and confining ovulation, the problem of determining the precise point when ovulation has occurred is of ever-increasing interest throughout the world, both in the animal husbandry industry and with respect to humans. It is especially important for human couples suffering from infertility to determine if and when ovulation occurs, to maximize the potential for pregnancy. At ovulation, an egg is released from the ovary. This egg remains viable approximately 12-24 hours. Conception occurs when an egg, present in the fallopian tube, is fertilized by a sperm. However, the egg is present in the fallopian tube typically for only a few hours, usually from 3-10. Meanwhile, sperm remains viable in a female for up to approximately 48 hours. Thus, if a woman wishes to become pregnant it is necessary for the sperm and egg to interact within the 12-24 hours preceding ovulation.

Meanwhile, for other couples who desire to practice family planning or minimize the potential for pregnancy by monitoring the female ovulation cycle, it is possible to determine that portion of the month where conception is physically impossible. Thus, both infertility and birth control may be impacted by the ovulation cycle.

There are many types of tests known in the art that can be conducted for the purpose of predicting the time of ovulation. For example, one well-known test is designed to predict ovulation by comparing the pH in a woman's saliva with that of her vagina over the course of time. By comparing the changes in the pH levels of a woman's body, it is scientifically possible to predict when ovulation will occur. Such a test employs a device similar to a digital pH meter, which permits a determination of the pH of a solution. Similarly, tests are well known for measuring the level of luteinizing hormone in various body fluids, as a predictor of ovulation occurrence. These tests typically either include a urine test or a blood test. Luteinizing hormone is a hormone of protein-carbohydrate composition that is obtained from the interior lobe of the pituitary gland, and that in the female, stimulates the development of corpora lutea, and together with follicle stimulating hormone, the secretion of progesterone. Other methodologies of the prior art rely on the concept that it is possible to predict when ovulation has occurred, based on the well-known basal body temperature increase in a female (up to 1 degree Fahrenheit) shortly after the time of ovulation. Still another methodology involves “ferning” of the cervical mucus. The optimal time for conception is thought to be when the cervical mucus is at its maximum density. Medical personnel, in conjunction with a pelvic exam, can examine mucus which has been deposited on a slide under a microscope to determine the amount of “ferning.” Whereas the normal amount of “ferning” ranges from 1-3, optimal conditions for conception exist when the value associated with “ferning” is 4+.

Attempting to conduct all of the methodologies described above can be time consuming, relatively expensive, and requires the assistance of medical professionals. However, to verify that optimum conditions exist, in the usual case all of the above tests should be performed and should test positive.

The need for a simple but reliable method of predicting and confirming ovulation, which can be conveniently carried out in the privacy of the home, has been a recognized need for many decades. Because of religious, philosophic, or health considerations, the preferred method of birth control for many is by periodic abstinence, also known as the “rhythm method.” This method involves the identification of the fertile period using an available method, or more often simply by a guess based on the length of the menstrual cycle, and then avoiding coitus during this period. Ovulation is assumed to occur mid-cycle, and the period of abstinence is adjusted accordingly. This technique has proven highly unreliable at best. The unreliability of the rhythm method is largely due to the inability to accurately predict and confirm ovulation. Thus, a clear need exists for a natural family planning method, acceptable under existing cultural and religious mores, which offers improved reliability without the need for intervention by medical professionals.

To address some of the foregoing issues, a more reliable procedure, known as the sympto-thermal method, involves a subjective evaluation of both basal body temperature and cervical mucus to determine the fertile period. However, this method requires intensive user training, and relatively high variability of results and failure rates were and are still unavoidable. In this procedure, changes in cervical mucus are combined with basal body temperature (“BBT”) to identify the onset and end of the fertile period. There are several disadvantages with this approach, among them being the need for immobility before taking the BBT, daily monitoring of the cervix and vagina, and subjective interpretation of vaginal mucus quality and of the BBT trend. The technique is difficult to learn, with one to six months of careful training and supervision being required to attain proficiency. Another relatively serious problem with this particular methodology is the variation of the relation between the basal body temperature and the peak mucus symptom. Furthermore, BBT correlates with ovulation in only about 70% of female cycles, since monophasic (non-indicative) BBT patterns are frequently seen in ovulatory cycles. Thus, when use of this methodology has been attempted for birth control, failure rates of up to 34% have been recorded. Although computerized interpretation of data is now available for the sympto-thermal method, for example as disclosed in U.S. Pat. No. 4,151,831, issued May 1, 1979, to Lester, the disadvantages obviously inherent in the physiological parameters used in the method are still limiting factors.

Other methods well known in the art are more invasive of the body, but can be good indicators of the time of ovulation. The identification of a rise in estrogens prior to ovulation followed by a peak in luteinizing hormone (LH) concentration, as determined by radioimmunoassay, is a good indication of imminent ovulation. Frequently, several samples of blood, drawn at mid-cycle, are analyzed for luteinizing hormone concentration. These techniques are expensive, involve invasive drawing of blood from the subject, and require several visits to a hospital or medical laboratory having the appropriate analytical facilities.

The process of ovulation has also been monitored and detected using ultrasonography. However, this procedure is time-consuming and expensive for the subject, as daily visits to a center equipped with the sophisticated instrumentation used for the procedure are necessary. Several scans are required to pinpoint ovulation by observing follicular development and subsequent ovum release. While accurate identification of ovulation is possible with this technique, it is of little value as a self-monitoring method for purposes of enhancing or reducing fertility.

Several methods of predicting ovulation based on biochemical changes in various body fluids such as vaginal secretions, saliva, or urine have been proposed. The major drawback of such methods is the significant variation in the component being measured between individuals. For example, in one method, where the lactic acid concentration of vaginal secretions was proposed as an indicator of impending ovulation, the variability of its concentration between individuals was as great as one thousand percent. See U.S. Pat. No. 4,010,738, issued Mar. 8, 1977, to Preti et al.

As a practical matter, it is generally believed at the present time to be impossible to design a chemical indicator system that is applicable to all women. One example of such a problem is related to the alkaline phosphate levels of saliva that, although appearing to be predictive of ovulation, show such variations among individuals that any chemical means of monitoring for its changes requires that tests be individually calibrated to each user. Methods based on analysis of urine for steroid hormones or their derivatives are subject to the same problem.

U.S. Pat. Nos. 4,685,471, 4,770,186 and 4,836,216 describe methods and apparatus for predicting ovulation in a human female subject which overcome many of the shortfalls and limitations of the methods described above. In the performance or this method, the onset of menstruation of the subject is noted, and daily determinations are made of the electrical resistivity of the subject's saliva, beginning not more than five days following beginning of menstruation. The onset of ovulation is determined as a function of a peak resistivity measurement following onset of menstruation. Vaginal resistivity measurements may be made to confirm ovulation. A probe is used for the resistivity measurement. In addition, U.S. Pat. No. 6,364,844 to Regas, et al., issued Apr. 2, 2002, and commonly assigned herewith, discloses improvements in a diagnostic system for determining and/or monitoring a physiological condition in a female mammal are disclosed. The disclosed system comprises: (a) a read only memory means for storing a diagnostic program for determining the fertility state of the female subject from salivary and vaginal resistance data values, (b) a random access memory means for storing temporary data values; (c) a non-volatile memory means for storing daily data values; (d) a display means for displaying characters representing the fertility state; (e) a microprocessor means for controlling the processing of data in accordance with said diagnostic program, said microprocessor means being connected to read only memory means, said random access memory means, said nonvolatile memory means, and said display means; (f) a sensing means for sensing the resistance of an electrical path along the tongue or vagina of the female user and outputting analog signals, (g) a conversion means for converting said analog signals to digital signals; (h) an input means for enabling the input of data by the female subject; and (i) a first interface means connected to said input means and said analog-to-digital conversion means for enabling the input of digital signals representing a current daily data value to said microprocessor means. The diagnostic program comprises algorithms for the recognition of predetermined patterns of data values, said algorithms being applied to said current daily data value and said stored daily data values by said microprocessor means, said microprocessor means controlling said display means to display characters representing one of several fertility states of said subject in response to recognition of a corresponding one of said predetermined patterns. The improvements involve the algorithms being capable of determining, substantially automatically, variables that can be used to define the beginning of the fertile cycle of the mammal, the end of the fertile cycle of the mammal and the most fertile day of the mammal.

Although the methods and apparatus described in the four patents discussed in the paragraph above are greatly advantageous over all of the methodologies previously described, nevertheless there is a need for continuous improvement, in order to provide more accurate results and increase the subject's compliance with the proper procedures for performing the methods, provide greater ease of use and also to increase acceptance of these improved methods and apparatus by philosophical and religious groups.

Furthermore, in the period of time during which the above-described methodologies have been part of the art, a paradigm shift in the way fertility data should be presented to the user has occurred, due to the requirements of the present-day consumer. Current commercially-available methodologies for presenting fertility data are based on the user receiving a level of fertility displayed on a scale specific to the device being used. For example, a device in accordance with the disclosure of U.S. Pat. No. 6,364,844 to Regas, et al., issued Apr. 2, 2002, as described above, displays a fertility status on a progress bar with levels from 1-7 bars, 7 bars representing the highest fertility. Another device, the Clearblue Easy (commercially available from Inverness Medical) presents fertility status on a 3 bar level: low, possible and highest fertility. However, in the case of both of the foregoing devices, while the devices are useful, the data is presented for the current day only. The improved apparatus and methods of the present invention seek to increase the amount of data available to the user of a device in order to provide an improved methodology to achieve the above-described advantages.

### SUMMARY OF THE INVENTION

Accordingly, in accordance with the present invention, in one aspect of the present invention, an improved diagnostic system is provided comprising an improvement in said U.S. Pat. No. 6,364,844 to Regas, et al., issued Apr. 2, 2002, and incorporated herein in its entirety by reference, wherein a user interface is provided for the system which further comprises a means of display of the physiological condition over time to the user.

In a particularly preferred embodiment, there is also provided by the present invention an improved ovulation prediction apparatus based on that disclosed and claimed in the aforementioned commonly-assigned patent, and having a housing which contains electronic means for processing the above-described information, and also preferably including means associated therewith which serve as one or more sensors to transmit information to the means for processing information, and a means for substantially automatically indicating the presence of a viable egg, including an audible component and a visual component. The means for processing the information includes at least one microprocessor-controlled circuit. The information is processed and compared with data associated with the presence of a viable egg, such that if a viable egg is empirically indicated as being present, then the means for indicating the presence of a viable egg will indicate such presence.

Accordingly, it is a primary object of the present invention in a preferred embodiment to provide an improved electronic ovulation prediction apparatus having the above-described user interface providing the improvements described, that is simple and convenient to use, and results in a quick and effective determination of the time of ovulation displayed over a period of time, and confirmation of whether a viable egg is present at the optimum time for insemination.

It is a further object of the present invention to provide novel, additional, and greatly advantageous uses and applications which are enabled by the improved ovulation prediction apparatus and the method for utilizing it of the present invention, which have been discovered to be outside the field of ovulation prediction. Accordingly, it is to be understood and appreciated that these discoveries in accordance with the invention are only those that are illustrative of the many additional potential applications of the apparatus and method that may be envisioned by one of ordinary skill in the art, and thus are not in any way intended to be limiting of the scope of the invention. Other objects and advantages of the invention will be apparent to those skilled in the art from the following detailed description of the invention.

### BRIEF DESCRIPTION OF THE DRAWINGS

FIG. 1 shows system electrical components of a device in accordance with a preferred embodiment of the present invention.

FIG. 2 shows a “Boot” screen of a device in accordance with a preferred embodiment of the present invention.

FIG. 3 shows a “Calendar” screen of a device in accordance with a preferred embodiment of the present invention.

FIG. 4 shows a “Menu” screen of a device in accordance with a preferred embodiment of the present invention.

FIG. 5 shows a “Settings” screen of a device in accordance with a preferred embodiment of the present invention.

FIG. 6 shows a “Readings” screen of a device in accordance with a preferred embodiment of the present invention.

FIG. 7 shows a “Connect And Place Sensor” screen of a device in accordance with a preferred embodiment of the present invention.

FIG. 8 shows a “Taking Oral Reading” screen of a device in accordance with a preferred embodiment of the present invention.

FIG. 9 shows an “Oral Data” screen of a device in accordance with a preferred embodiment of the present invention.

FIG. 10 shows a “Bad Reading” screen of a device in accordance with a preferred embodiment of the present invention.

FIG. 11 shows a “Connect and Place Sensor” screen of a device in accordance with a preferred embodiment of the present invention.

FIG. 12 shows a “Taking Vaginal Reading” screen of a device in accordance with a preferred embodiment of the present invention.

FIG. 13 shows a “View Vaginal Data” screen of a device in accordance with a preferred embodiment of the present invention.

FIG. 14 shows a “Bad Reading” screen of a device in accordance with a preferred embodiment of the present invention.

FIG. 15 shows a “View Data” main screen of a device in accordance with a preferred embodiment of the present invention.

FIG. 16 shows an “Open Application” screen of a device in accordance with a preferred embodiment of the present invention.

FIG. 17 shows a “Connect USB” screen of a device in accordance with a preferred embodiment of the present invention.

FIG. 18 shows a “Downloading Data” screen of a device in accordance with a preferred embodiment of the present invention.

FIG. 19 shows a “Help” screen of a device in accordance with a preferred embodiment of the present invention.

### DETAILED DESCRIPTION OF THE INVENTION

Thus, in one aspect the present invention provides an improved diagnostic system for determining a physiological condition of a mammal, such as the onset of ovulation in a female mammal. The diagnostic system comprises improvements in the diagnostic system as disclosed and claimed in U.S. Pat. No. 6,364,844 to Regas, et al., issued Apr. 2, 2002, for determining and/or monitoring a physiological condition in a mammal, such improvements including providing a user interface for the system which further comprises a means of display of the physiological condition over time to the user. The improvements according to the present invention provide increased accuracy and reproducibility of the results of the methods disclosed in said patent, enabling advantageous results by comparison with this prior system as well as by comparison with previous methods of the art. By means of further research involving the use of the method and apparatus described in this patent, numerous additional and greatly advantageous applications for the method and apparatus have been discovered. It will therefore be appreciated that these improvements and discoveries have enabled the invention described and claimed herein to be greatly improved over all of the prior systems and methods, and to have applications for determining and/or monitoring various physiological conditions in all female mammals, other than providing merely the prediction and confirmation of ovulation.

### REFERENCE TO EXISTING PATENT

It will be appreciated that the improvements provided by the present invention have been made, in preferred embodiments, to the devices and methods disclosed in U.S. Pat. No. 6,364,844 to Regas, et al., issued Apr. 2, 2002, commonly assigned herewith. This patent, as described above, is expressly incorporated herein by reference.

According to the present invention, the apparatus and methods disclosed in the foregoing U.S. Pat. No. 6,364,844 are greatly and advantageously improved by the use of the discoveries and features of the invention, which are now described in more detail in the following text. These improvements, and novel applications of the thereby improved apparatus and methods of the invention, together with their advantages, will be readily apparent to one skilled in the art by reference to the above-described patent and the improvements to the invention disclosed and claimed therein as provided by the preferred embodiments of the present invention as described herein.

### Description of Preferred Embodiments

Thus, as has been described above, the present invention provides specific improvements to the prior apparatus and methods described in the above-listed, commonly assigned US patent. It is therefore to be appreciated that all of the uses, advantages and features of the inventions as disclosed and claimed in said incorporated US patent will apply to the present invention as well. In addition, these improvements, as set forth in the description of the present invention provided herein, as well as many other advantages over the apparatus and methods of the prior art, as described in the foregoing text, will be apparent to those skilled in the art. Specific preferred embodiments thereof will now be described in greater detail.

The preferred embodiments of the present invention described herein, which incorporate these improvements, make possible not only the improved features and ease of use of the apparatus and methods provided by the present invention for predicting and confirming ovulation in mammals, as described previously, but also have been found to enable the invention to be employed in many fields and applications in addition to the prediction and confirmation of ovulation, such as in the diagnosis, prevention and treatment of various physiological and disease states, and thus it is to be appreciated by those skilled in the art that the present invention is applicable to all of such physiological and disease states.

Accordingly, the improvements provided by the invention will now be described in terms of specific preferred embodiments, which are intended only for illustration of the present invention and are not intended to constitute limitations thereon.

In one especially preferred embodiment of the present invention, in contrast to methods previously known in the art, the present invention provides a novel method for presenting fertility data to a user, not only for a given day, but over time. According to the present invention this is accomplished by employing a set of unique software protocols which when employed for use with the hardware of a device in accordance with the invention, utilize a color coding system on an electronic calendar that can be accessed with simple arrow keys and an enter button of the device, being, for example, similar to the way a calendar is used on a cell phone. In this way a user can move to any month on a calendar and see fertility data for the entire month displayed on each day of the calendar as a different color representing the fertility status for that day. Higher fertility is coded with a darker color blue and actual ovulation is marked with a bright pink color. By showing fertility data over time via a color coded system, the user can view trends from month to month and easily ascertain, up to seven days in advance, when the time of highest fertility will be reached.

In another especially preferred embodiment of the present invention, a particularly novel feature of the improved method of this invention involves displaying fertility data, in such a way as to allow the user to press the enter button on the device on any given day and be presented with the current fertility status level from 1 to 7, if this is a most fertile day and the expected maximum fertility day(s) for the cycle. In a further preferred embodiment, once data is collected, the device of the present invention will also present future fertility predictions for the next cycle as color coded fertility on a calendar, but with the days slightly hashed to indicate a prediction, rather than a confirmation.

The means for collecting data from oral or vaginal sensors, in the method and device in accordance with the present invention, can be substantially that described in the foregoing U.S. Pat. No. 6,364,844 to Regas, et al., issued Apr. 2, 2002. However, in accordance with the improvements provided by the present invention, fertility data can be accessed and presented in a novel manner by use of a unique user interface that is not taught by the disclosure of this patent or by any known previous art. This methodology of the present invention significantly and advantageously extends the functionality of the invention into unique and substantially different capabilities, enabling the user, through the novel interface provided to access and view fertility data over time and in a substantially different manner than any previously known.

In a preferred embodiment, the user interface screens are graphical in nature, and can be similar to a calendar in a conventional cell phone, with the month displayed across the top of the screen. Different months can be accessed with the left and right arrow keys of the device, the right arrow moving forward through the months and the left moving to earlier months. In use by a woman who is monitoring her fertility cycle, this functionality enables the woman to see her fertility data over time. In addition, the up, down, left and right arrows, can then be used to access the fertility data stored for each individual day. By moving to the day of interest and pressing the center button (enter key), the user can view more detailed fertility information for that particular day. In a particularly preferred embodiment of the present invention, the fertility status can be presented to the user of the device by means of a graph of 1-7 bars and by color, wherein the expected maximum fertility days and the particular day typically of most interest, the day of highest fertility, are all displayed in the day detail accessed by this feature.

Specific embodiments of the improvements provided by the present invention are set forth in the following description, which is provided to exemplify the present invention in some of its preferred embodiments.

In order to implement the improvements provided by the present invention, novel hardware, and software specifically designed for use with such hardware, has been utilized. Such hardware and software, in combination, advantageously enables the novel user interface features of a device that has been improved by the present invention, as described above. The novel hardware and software is described below in connection with particularly preferred embodiments of the invention.

Definitions/Abbreviations
MCU—Microprocessor Control Unit
STM—STMicroelectronics
SPI—Serial Peripheral Interface
UART—Universal Asynchronous Receiver Transmitter
USB—Universal Serial Bus
TFT—Thin Film Transistor
LCD—Liquid Crystal Display
MBIT—Mega Bits

In one example of a preferred embodiment of the present invention, the improvements provided thereby are utilized in a device that is intended to be used by human consumers to help with the conception of a child. The device will typically comprise a handheld unit to determine the maximum fertility of the user. The device has a large LCD screen and a seven button interface, and is designed so that it is capable of being comfortably held in one hand. The device has two buttons (oral and vaginal) to easily take readings, and the other buttons are used to navigate through the menu system. In a particularly preferred embodiment, the menu system of the device is icon based, and the main screen is a calendar, on which the user will be able to navigate and display the fertility information for that day.

It is preferred that the device be able to record and store previous readings, which are then used in conjunction with the algorithms described in U.S. Pat. No. 6,364,844 to Regas, et al., issued Apr. 2, 2002, incorporated herein by reference, to enable obtaining of the best possible fertility date. The system preferably also has a USB interface to download the data to a computer using the software and graph and manipulate the data.

It is also preferred that the device have two sensors, one oral and in addition an (optional) vaginal sensor. The oral sensor is intended to be used for everyday use, while the vaginal sensor is intended to be used for a more accurate reading, if deemed necessary by the user.

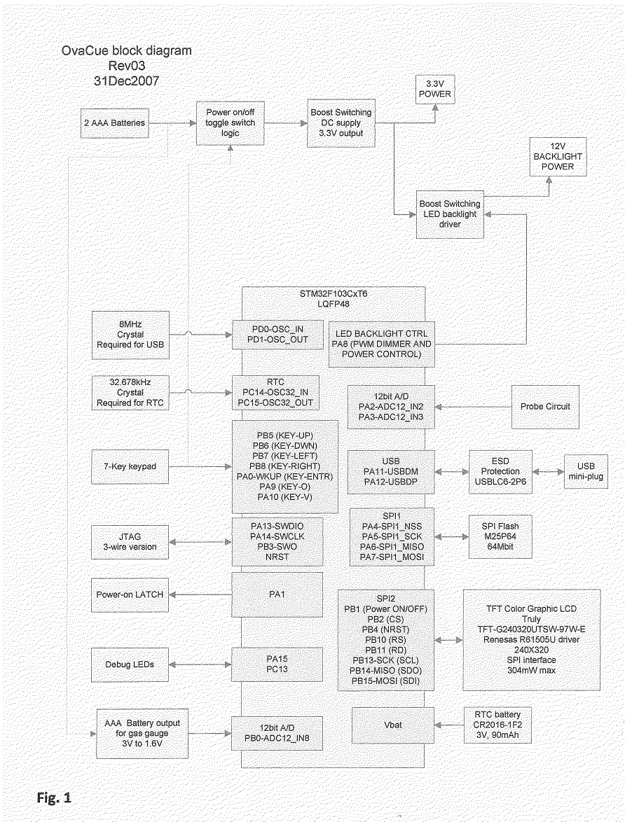

Referring now to the drawings, FIG. 1, below, shows the block diagram of the system electrical components of a preferred embodiment of a device that has been improved in accordance with the present invention.

MCU PCB

The MCU printed circuit board hosts the MCU STM module and provides control to the other components in the device.

STM Module

The STM32F103Cx incorporates the high-performance ARM Cortex-M3 32-bit RISC core operating at a 72 MHz frequency, high-speed embedded memories (Flash memory up to 128 Kbytes and SRAM up to 20 Kbytes), and an extensive range of enhanced I/Os and peripherals connected to two APB buses. The device has two 12-bit ADCs, three general purpose 16-bit timers plus one PWM timer, as well as standard and advanced communication interfaces: up to two I2Cs and SPIs, three USARTs, a USB and a CAN.

USB Module

The USBLC6-2P6 is dedicated to ESD protection of high speed interfaces for USB2.0, and is backward compatible to USB1.0. The very low line capacitance secures a high level of signal integrity without compromising in protection sensitive chips against the most stringent characterized ESD strikes.

Flash Memory Module

The M25P64 is a 64 Mbit (8M×8) Serial Flash Memory, with advanced write protection mechanisms, accessed by a high speed SPI-compatible bus. The memory can be programmed 1 to 256 bytes at a time, using the Page Program instruction

Power Supply

It is preferred that the device contain one power supply, preferably based on two AAA batteries. The system monitors the power and a graphical representation of the remaining power will be displayed to the user on the LCD.

Keypad

The device has seven keys, two hot keys and five menu keys. The two hot keys are used to easy take an oral or vaginal reading without navigating through the menus to take a reading. The menu keys are used to move up, down, left, right, and enter through the menus. To turn the system on and off hold the enter key for 3 seconds. See Table 1 below.

TABLE 1
Key Options (X - Available, ◯ - Unavailable)

| | Up | Down | Left | Right | Enter | Oral | Vaginal |
|---|---|---|---|---|---|---|---|
| Menu | Key | Key | Key | Key | Key | Key | Key |
| Boot | ◯ | ◯ | ◯ | ◯ | ◯ | ◯ | ◯ |
| Calendar | X | X | X | X | X | X | X |
| Settings - Main | X | X | X | X | X | X | X |
| Readings - Main | X | X | X | X | X | X | X |
| Readings - | X | X | ◯ | ◯ | X | X | X |
| Connect Oral | | | | | | | |
| Sensor | | | | | | | |
| Readings - | X | X | ◯ | ◯ | X | X | X |
| Taking Oral | | | | | | | |
| Reading | | | | | | | |
| Readings - Oral | X | X | X | X | X | X | X |
| Data | | | | | | | |
| Readings - | X | X | ◯ | ◯ | X | X | X |
| Connect vaginal | | | | | | | |
| Sensor | | | | | | | |
| Readings - | X | X | ◯ | ◯ | X | X | X |
| Taking Vaginal | | | | | | | |
| Reading | | | | | | | |
| Readings - | X | X | X | X | X | X | X |
| Vaginal Data | | | | | | | |
| Readings - View | X | X | X | X | X | X | X |
| Data Main | | | | | | | |
| Readings - View | X | X | ◯ | ◯ | X | X | X |
| Data | | | | | | | |
| Readings - | X | X | ◯ | ◯ | X | X | X |
| Connect USB | | | | | | | |
| Readings - | X | X | ◯ | ◯ | X | X | X |
| Downloading | | | | | | | |
| Help - Main | X | X | ◯ | ◯ | X | X | X |

Liquid Crystal Display

The display is a TFT-LCD from Truly Semiconductors with a built in Rensas control driver. The resolution of the display is 240×320 pixels with an internal backlight. The display can handle up to 262K colors. The display supports parallel, serial, and RGB interfaces.

System Software

It is to be appreciated that in a particularly preferred embodiment, the diagnostic system of the invention is incorporated in a handheld device, such as that described in detail in the hereby incorporated-by-reference disclosures of U.S. Pat. Nos. 4,685,471, 4,770,186, 4,836,216, and No. 6,364,844, the hardware of the improved device and system provided by the invention can be further enhanced by the use of software to provide functionality previously not possible in a hand-held apparatus of this type.

Initialization

The initialization function performs a one-time initialization and setup for the processor hardware. The ports are configured for input or output. The real-time clock is restored and the system timing initialized.

Configuration

The configuration function is called once on startup after initialization. Configuration consists of the initialization of software sub-systems and data structures. The NCMCU configuration data is stored in non-volatile memory, and contains the following data:
Version—the version of the configuration structure
Serial Number—The serial number of the device

Main Loop

Once initialized and configured, the software will execute the main loop until the processor is reset or turned off. The main loop will perform the following duties:

NC Process

The NC process forms the interface between the firmware and the device input/output. Ports are read and the LED outputs are updated in this routine.

USB Process

The USB process routine will scan the USB port and determine if a cable is connected; if the user selects the download icon, the system will transfer the fertility records to a personal computer (PC) connected to the device via the USB port.

State Process

The main operation of the MCU will be implemented as a state machine. The state machine will be called once per main loop. Based on the current state of the device (off, calendar menu, oral reading, etc), the software will be dispatched to the matching state processing routine. States will be implemented as a series of sub-states and sub-sub-states.

Flash Process

The flash process will limit the storing of changes to the configuration and records to non-volatile memory. Whenever the configuration is changed or a record needs to be saved, the structure will be marked dirty. Whenever the configuration or record is dirty and the configuration or record write timer has expired, the configuration or record will be written to non-volatile memory.

Profile

Profile will measure the elapsed time in milliseconds from the beginning to the end of the main loop. If the time spent in the main loop is longer than the previous times, the duration will be printed to the debug display.

Power Process

The power process checks to see if the power saver timer has elapsed, which will turn the system off or go into sleep mode to save power. The timer will be reset every time a button is pressed or an ADC reading has changed. The system will turn the backlight off after 30 seconds and will go into power saver mode after 2 minutes.

It will be appreciated that he application logic of the improvements provided in the user interface functionality of the novel device in accordance with the present invention, can be implemented as a multiple level state machine. The state machine organizes processing by application state (Off, Boot, Calendar Menu etc.). Each main state of operation is sub-divided into a series of sub-states. In addition, these sub-states are often divided into independent sub-sub-states. The states are processed through a single routine that dispatches to the appropriate subroutine based on the current state and sub-state values.

The top-level states are:

Boot—The device has been turned on and is initializing

Calendar—The device is in the main menu showing a calendar and date in formation.

Settings—Settings for the system like the system time and other information

Readings—Take oral or vaginal readings, view data, and download data

Help—Show the company information and contact info.

Boot State

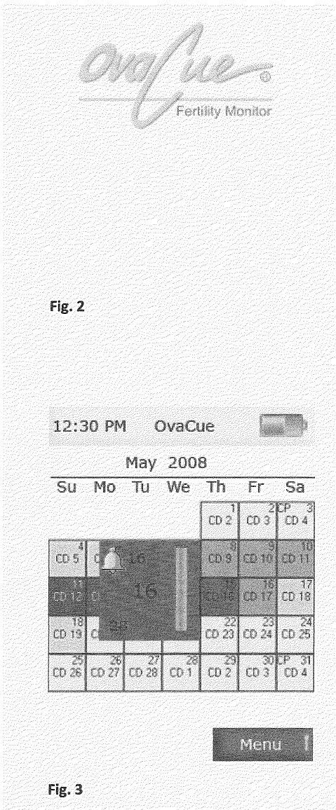

When the device is turned on or reset, the device enters the boot state. FIG. 2, below, shows a typical layout for the boot screen in a preferred embodiment of a device in accordance with the invention.

Calendar

The calendar screen is the main screen for the system. This screen allows the user to navigate through the days by hitting the up, down, left, and right buttons. If the is user hits the enter button on a certain day then the record for that day will be displayed. The current time and power meter will be on the top of the screen. Along the bottom is the menu button to access the sub menus. FIG. 3, below, shows a typical Calendar Screen in accordance with a preferred embodiment of a device in accordance with the present invention.

Menu

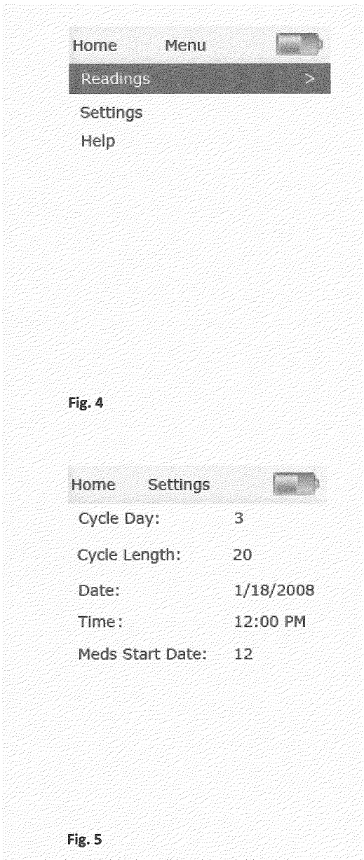

The settings screen will have a list of the sub menus available. The user and navigate through the list and a blue box with white text will show the user what item they are on. By pressing the enter key the system will go to the next menu. If the user hits the up button on the top list item the blue box will jump up to the Home button in the top left corner. If the user hits enter when the Home button is highlighted then the system returns to the Calendar screen. This feature is preferably present in all screens but the Calendar. FIG. 4, below, shows the layout of a typical menu screen in a preferred embodiment.

Settings

The settings screen will display the cycle length, cycle start date, date, time, and the medication used. The user can scroll up and down and press the enter key to change the selected value. After the value has been changed, another press of the enter key will save the value. FIG. 5, below, shows the layout of the setup menu screen in a preferred embodiment.

Readings

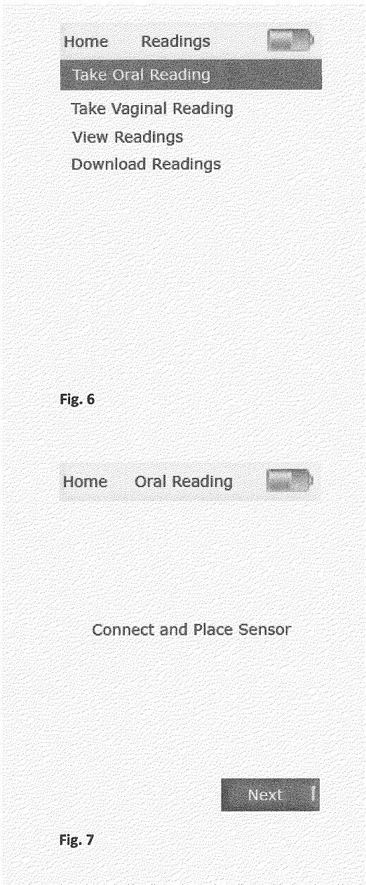

When the user clicks the Readings in the Menu the Readings sub menu will be displayed. A list of the sub sub menus will be displayed allowing the user to scroll up and down selecting the task they want to accomplish. The home button will redirect the system back to the calendar screen. FIG. 6, below, shows the Readings Screen.

Oral Readings

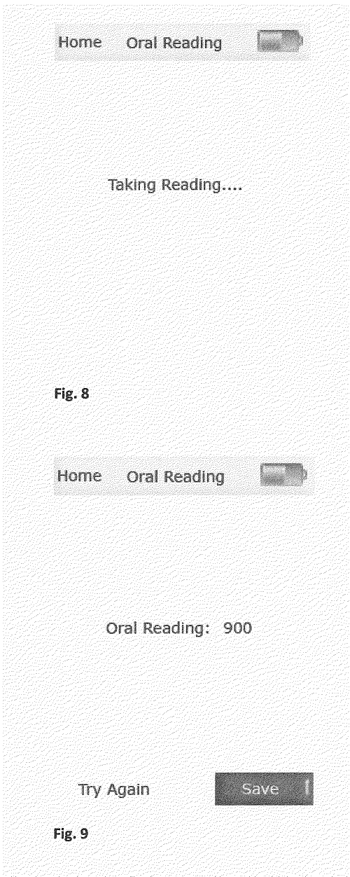

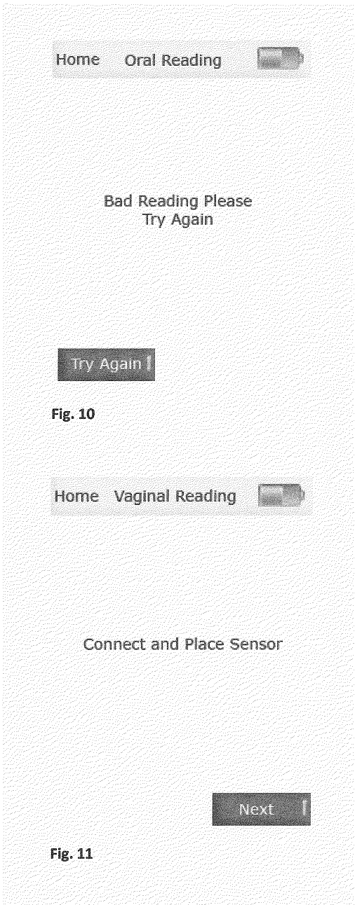

When the user clicks the Take Oral Reading in the Reading Menu or presses the oral speed key the Connect and Place sensor screen will be shown. If the user clicks the next button the system will go to the taking reading screen. Once the reading is finished the information will be displayed to the user with a save or delete button. A bad data screen will appear if the reading is above 400. If the save button is clicked the information will be saved for that day and return to the Calendar screen. If the try again button is selected the system will return to the Connect and Place Sensor screen. FIGS. 7-10, below, show the Oral Reading screens.

Vaginal Reading

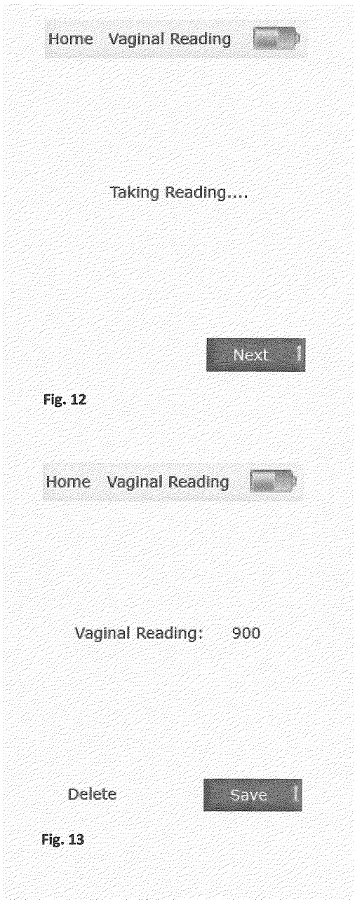

When the user clicks the Take Vaginal Reading in the Reading Menu or the vaginal speed key, the Connect and Place sensor screen will be shown. If the user clicks the next button the system will go to the Taking Reading screen. Once the reading is finished the information will be displayed to the user with a save or delete button. A bad data screen will appear if the reading is above 400. If the save button is clicked the information will be saved for that day and return to the Calendar screen. If the Try Again button is selected the system will return to the Connect and place Sensor screen. FIGS. 11-14, below, show the Vaginal Reading screens.

View Data

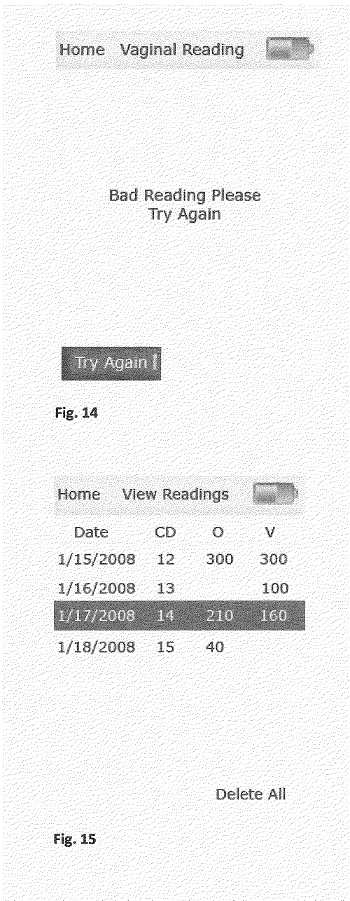

When the user clicks the View Data icon on the Calendar Menu the main View menu will be shown. If the user clicks the View Fertility Data icon the data summary menu will appear. This screen will allow the user to view all of the records on the system. The user will be able to scroll through the records and view or delete the selected record. FIG. 15, below, shows the layout of the View Data screens.

Download Data

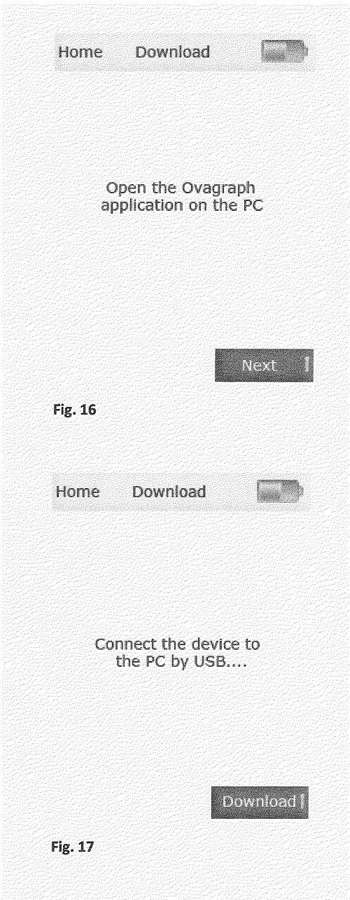

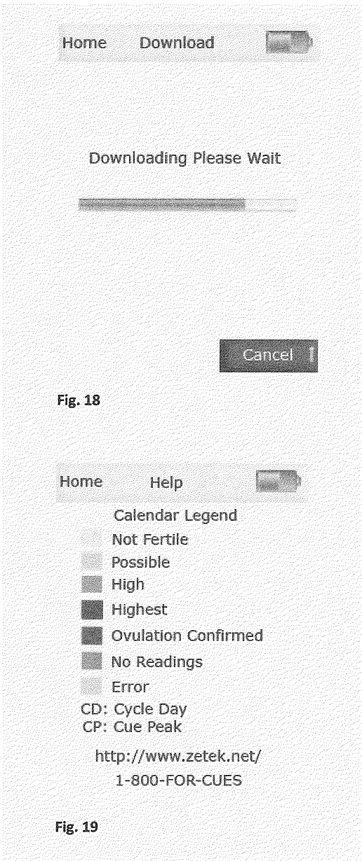

When the user clicks Download in the Readings Menu the open Ovagraph application screen will be shown. The user then clicks next after the application is up and running and the Connect USB screen will be displayed. The user then clicks the Download button to start the download. The downloading screen will appear with a progress bar to indicate how much data has been downloaded and a cancel button. After the download has completed or the cancel button was pressed the system will return to the calendar menu screen. FIGS. 16-18, below, show the layout of the Download screens.

Help

The Help screen will display the legend for the Calendar screen and also the contact information for the manufacturer. FIG. 19, below, shows the layout of the Help screen.

Libraries

The firmware included in a preferred embodiment of a device in accordance with the present invention can utilize a series of libraries specific to its development in addition to the conventional standard, which is a C run-time library.

USB Communications

The USB library, in a preferred embodiment, can have a custom protocol to transfer data samples from the device to a PC that had been connected to the device. The library can include functions for initializing, sending, and receiving characters. A series of utility functions also can be provided to simplify repetitive tasks.

LCD Library

The LCD library can be used to communicate to the LCD to draw the menus and graphics on the screen. Preferably, the library can include functions for initializing, clearing, and display characters on the screen. A series of utility functions also can be provided to simplify repetitive tasks.

Flash Library

The Flash library can be used to communicate to the flash module to write the fertility information. The library preferably can include functions for initializing, unlocking, erasing, and writing data. A series of utility functions also can be provided to simplify repetitive tasks.

Algorithm Library

An algorithm library can provide the algorithm, for example, preferably an algorithm provided by the device manufacturer and as set forth in U.S. Pat. No. 6,364,844 to Regas, et al., issued Apr. 2, 2002, incorporated by reference herein, to determine the fertility of the reading and also the best fertility date in the fertility cycle of the individual cycle. These algorithms were developed, following extensive research, and are preferably incorporated in the device of the present invention as well as in prior apparatus and methods described in the foregoing commonly-assigned patent, and are based on determining the few key variables that can be used to define the beginning of the fertile cycle (selection of a dominant follicle), the end of the fertile cycle (the occurrence of ovulation) and the most fertile day of a woman.

Software Modules

Preferred software modules that can be utilized in conjunction with the present invention are as follows:

Profile

The profile code is used to measure the run-time of the main loop. The profile timer is initialized at the top of the main loop. At the end of the main loop, the execution duration is measured in milliseconds. If the measured duration of the main loop is longer than any previous measurement, the duration is displayed on the debug port.

Configuration

The configuration of the device is stored in a single structure. A simple checksum is stored with the configuration to verify that the memory image is valid when it is restored from flash. A version number is stored with the configuration to identify the format of the memory segment. A device serial number is also stored with the configuration.

Status

The status of the system is shared globally with the system state machine. The status maintains the current state and sub-state values. The status structure also maintains a software variable for each of the interfacing hardware points, allowing all inputs and outputs to be read or updated in a single routine for synchronized operation.

Fertility Data

The Fertility data of the MCU is shared globally with the system state machine. The Fertility data structure is the information gathered during a reading to be saved to flash. The structure includes information about the device, the fertility reading, the date and time of the reading, drug usage, day of cycle, and any other information about the reading.

It is to be appreciated that while the foregoing description sets forth preferred embodiments of the present invention, it is intended not to place limitations thereon, and that other aspects of the invention riot specifically set forth herein are described in detail in U.S. Pat. No. 6,364,844 to Regas, et al., issued Apr. 2, 2002, which is incorporated herein by reference, and that the present invention provides many advantageous improvements over the invention disclosed and claimed in the foregoing patent, as will be appreciated from the description above.

Manufacturing:

In manufacturing of the improved diagnostic system of the present invention, a commercially available, high-integration microcontroller with built-in time codes, ROM and improved firmware capabilities is used, together with an improved microprocessor. As described above, an enhanced, substantially automated capability is provided by the algorithm for determining peak fertility based on the results of the readings. In the manufacturing process, the algorithm is developed and compiled for implementation into the microprocessor contained in the device, in a manner well known to those skilled in the art. This enables the device to substantially automatically interpret the data, thereby providing the user with almost immediate and clear results.

As will be apparent to those skilled in the art, the improvements provided by the instant invention are not to be limited by the foregoing description of preferred embodiments, and any such limitations are only to be construed as being defined by the claims appended hereto.

What is claimed is:

1. In a diagnostic system including a user interface for determining or monitoring a physiological condition in a mammal, said diagnostic system comprising:
(a) a read only memory means for storing a diagnostic program for determining the fertility state of the mammal from salivary and vaginal resistance data values,
(b) a random access memory means for storing temporary data values;
(c) a non-volatile memory means for storing daily data values;
(d) a display means for displaying characters representing the fertility state;
(e) a microprocessor means for controlling the processing of data in accordance with said diagnostic program, said microprocessor means being connected to read only memory means, said random access memory means, said nonvolatile memory means, and said display means;
(f) a sensing means for sensing the resistance of an electrical path along the tongue or in the vagina of the female mammal and outputting analog signals,
(g) a conversion means for converting said analog signals to digital signals;
(h) an input means for enabling the input of data; and
(i) a first interface means connected to said input means and said analog-to-digital conversion means for enabling the input of digital signals representing a current daily data value to said microprocessor means,
and said diagnostic program comprising algorithms for the recognition of predetermined patterns of data values, said algorithms being applied to said current daily data value and said stored daily data values by said microprocessor means, said microprocessor means controlling said display means to display characters representing one of several fertility states of said subject in response to recognition of a corresponding one of said predetermined patterns, and wherein said algorithms are capable of the automated determination of variables that can be used to define the beginning of the fertile cycle of the mammal, the end of the fertile cycle of the mammal and the most fertile day of the mammal;
the improvement wherein the user interface of the system further comprises a means of display of the physiological condition over time to the user.

2. The improved diagnostic system as defined in claim 1, wherein said algorithms are capable of the substantially automated determination of first, second, and third patterns of data values, said first pattern being that the current daily data value is less than the previous daily data value by a first predetermined amount, said second pattern being that the current daily data value is greater than the previous daily data value by a second predetermined amount and the previous daily data value is less than the next preceding daily data value by a third predetermined amount, and said third pattern being that the current daily data value is greater than the previous daily data value by a fourth predetermined amount.

3. The improved diagnostic system as defined in claim 1, further comprising means connected to said microprocessor means for converting parallel data signals to synchronous serial signals for forming a communications link with an external device.

4. The improved diagnostic system as defined in claim 1, further comprising a real-time clocking means operatively connected to said microprocessor means by way of a second interfacing means.

5. The improved diagnostic system as defined in claim 1, further comprising a decoding means connected to said microprocessor means for decoding control and address signals output by said microprocessor means.

6. In a diagnostic system including a user interface for determining and monitoring a physiological condition in a female subject, said system comprising:
(a) a first probe means for outputting data signals representing the resistance value of an electrical path along the surface of a first body part of said female subject;
(b) a second probe means for outputting data signals representing the resistance value of an electrical path along the surface of a second body part of said female subject;
(c) an electronic data processing means having a port for interchangeably receiving either of said probe means, said data processing means including
(i) means for applying a first pattern detection algorithm to said data signals in response to recognition of said first probe means,
(ii) means for applying a second pattern detection algorithm to said data signals in response to recognition of said second probe means,
(iii) means for outputting first and second control signals in response to detection of data signals representing first and second patterns of successive resistance values, respectively, and
(d) a display means connected to receive the output of said data processing means and to display characters representing first or second fertility states of said female subject, respectively, in response to receipt of said first or second control signals, said algorithms being capable of substantially automated determination of variables that can be used to define the beginning of the fertile cycle of the mammal, the end of the fertile cycle of the mammal and the most fertile day of the female subject; the improvement wherein the user interface of the system further comprises a means of display of the physiological condition over time to the user.

7. The improved diagnostic system as defined in claim 1, wherein the physiological condition is the onset of ovulation.

8. The improved diagnostic system as defined in claim 1, wherein the physiological condition is luteal phase defect.

9. The improved diagnostic system as defined in claim 1, wherein the physiological condition is luteinized unruptured follicle.

10. The improved diagnostic system as defined in claim 1, wherein the physiological condition is atresia.

11. The improved diagnostic system as defined in claim 1, wherein the physiological condition is anovulation.

12. The improved diagnostic system as defined in claim 1, wherein the physiological condition is Polycystic Ovary Syndrome.

13. The improved diagnostic system as defined in claim 1 or claim 6, wherein the physiological condition is an ovulatory disorder.

14. The improved diagnostic system as defined in claim 1 or claim 6, wherein the physiological condition is a non-ovulatory disorder.

15. In a method of diagnosing the existence of a physiological condition in a subject mammal, which method comprises the steps of making daily determinations of the electrical resistivity of the subjects saliva beginning not more than five days following the onset of menstruation by applying a first sensor for forming analog data signals representing salivary electrical resistivity, converting said analog data signals to digital data signals, processing said digital data signals over a predetermined time to obtain a digital data signal representing the current daily value of the salivary electrical resistivity, storing said daily salivary electrical resistivity values in memory, applying a first algorithm for pattern recognition to the stored values, generating a first recognition signal in response to the first occurrence of the current daily value being less than the previous daily value by more than a predetermined significant amount, displaying a signal indicating a high probability of conception in response to generation of said first recognition signal, digitally displaying said current value resulting from said processing step, applying a second algorithm for pattern recognition to the stored values, and generating a second recognition signal in response to the occurrence of a predetermined relation between three consecutive daily values, making daily determinations of the electrical resistivity of the subject's vaginal mucus beginning after the generation of said first recognition signal by applying a second sensor in the subject's vagina for forming analog data signals representing vaginal electrical resistivity, converting said analog data signals to digital data signals, processing said digital data signals over a predetermined time to obtain a digital data signal representing the current daily value of the vaginal electrical resistivity, storing said daily vaginal electrical resistivity values in memory, applying a third algorithm for pattern recognition to the stored values, generating a third recognition signal in response to the first occurrence of the current daily value being greater than the previous daily value by more than a predetermined significant amount, and displaying a signal indicating a low probability of conception one day after the generation of said second recognition signal, and wherein said algorithms are capable of substantially automated determination of variables that can be used to define the beginning of the fertile cycle of the mammal, the end of the fertile cycle of the mammal and the most fertile day of the mammal, and displaying the results of the foregoing steps on a user interface, the improvement wherein said user interface provides display of the physiological condition over time to the user.

16. The method of claim 15, wherein the physiological condition is the onset of ovulation.

17. The method of claim 15, wherein the physiological condition is luteal phase defect.

18. The method of claim 15, wherein the physiological condition is luteinized unruptured follicle.

19. The method of claim 15, wherein the physiological condition is atresia.

20. The method of claim 15, wherein the physiological condition is anovulation.

21. The method of claim 15, wherein the physiological condition is Polycystic Ovary Syndrome.

22. The method of claim 15, wherein the physiological condition is an ovulatory disorder.

23. The method of claim 15, wherein the physiological condition is a non-ovulatory disorder.
